(19) United States
(12) Patent Application Publication
(10) Pub. No.: US 2025/0057434 A1
(43) Pub. Date: Feb. 20, 2025

(54) Microwave Imaging for Breast Cancer Detection

(71) Applicant: Choon Sae LEE, Dallas, TX (US)

(72) Inventor: Choon Sae LEE, Dallas, TX (US)

(21) Appl. No.: 18/809,071

(22) Filed: Aug. 19, 2024

Related U.S. Application Data
(60) Provisional application No. 63/533,484, filed on Aug. 18, 2023.

(51) Int. Cl. A61B 5/0507; A61B 5/00

(52) U.S. Cl. CPC A61B 5/0507; A61B 5/742

(57) ABSTRACT

A novel imaging technique is introduced at low microwave frequencies. Since the frequency is low, there is virtually no harmful radiation effect. On the other hand, a wave does not penetrate a lossy biological medium when the frequency is low. In accordance with principles of the invention, a standing-wave concept is used to overcome such obstacle so that two contradictory conditions are satisfied simultaneously: high penetration depth and high resolution.

### CROSS-REFERENCE TO RELATED APPLICATIONS

This application claims the benefit of U.S. Provisional Application No. 63/533,484, filed Aug. 18, 2023, which application is hereby incorporated herein by reference, in its entirety.

### TECHNICAL FIELD

The invention relates generally to a system and method for detection of breast cancer and, more particularly, to a system and method for imaging using non-ionizing microwave technology.

### BACKGROUND

Breast cancer is the most diagnosed and deadliest among all cancer types. In 2020, there were 2.3 million women diagnosed with breast cancer and 685,000 deaths globally out of which 43,700 women died from breast cancer in the United States. Women whose breast cancer is detected at an early stage have a 93% or higher survival rate in the first five years. However, early detection techniques continue to have significant diagnostic challenges and thus remains the main focus of attention of a number of medical research groups. Well-established detection techniques classified as ionizing radiation (e.g., mammography) remain limited in their sensitivity to small-volume disease, image contrast, and background interference such as increased tissue density or scarring from prior surgery. Other functional imaging techniques, such as Magnetic Resonance Imaging (MRI) and Positron Emission Tomography (PET), have been used as a complement to anatomical imaging; however, the high cost and complex operational logistics contribute into their practical limitation for use in large-scale.

The interaction of electromagnetic signals with matter depends on the material's dielectric properties, such as the electric permittivity and conductivity. For body tissues, the dielectric properties are related directly to the water content of different biological constituents. Low water content tissues include fat and bones, whereas high water content tissues include muscles, brain, blood, internal organs, and tumors. Microwave imaging for breast cancer detection can be classified into two major categories: passive and active techniques. In passive systems, electromagnetic radiation emitted by living tissues is measured. This technique depends heavily on the difference in temperature between healthy and cancerous tissues. The main challenge to this method is to detect a very low-level power radiated by tumors, which raises complexity on technical design. On the other hand, in active systems, an electromagnetic signal is transmitted from a source into the tissues, and the reflected signals are measured. Two main active detection methods are mainly tomography and radar-based imaging. A drawback of a tomography method is lack of imaging accuracy, specificity, and high cost. Radar-based approaches use ultra-wideband (UWB) signals to satisfy the resolution requirement while maintaining adequate signal penetration as tissue conductivity increases with frequency. UWB has a better advantage compared to other techniques in which it can detect as small as 5 mm in addition to introduction of 3D imaging possibility. However, most of the feasibility studies carried out so far for such technique were performed through numerical simulations only and showed technical challenges.

The major problems with the UWB method are that it takes too much time for data collection and the penetration of the electromagnetic (EM) waves into the lossy biological tissues is severely limited at microwave frequencies.

In view of the foregoing, what is needed is a novel diagnostic method for breast cancer that uses non-ionizing microwaves, providing a portable, cost-effective imaging tool.

### SUMMARY

The proposed imaging method is a technique wherein the reported limitations can be bypassed by using standing waves in an enclosed volume as shown in FIG. 2 in comparison with a conventional radar-based microwave imaging technique that is relying on traveling waves.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows a conventional radar-based microwave imaging system according to the prior art;

FIG. 2 exemplifies a system using antenna cavity and standing waves in enclosed volume;

FIG. 3 exemplifies microwave imaging in a boxed structure; and

FIG. 4 exemplifies an alternative embodiment of the structure of FIG. 3.

The resulting images will show not only variation of the dielectric constant but also the electric conductivity of the tissue material, revealing blood vessels around tumorous areas. Such detailed images will help doctors in early cancer detection without adverse side effects to patients.

### DETAILED DESCRIPTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein. Additionally, as used herein, the term “substantially” is to be construed as a term of approximation.

In conventional microwave imaging devices, traveling waves are emitted by multiple transmitters and the scattered fields are measured and processed for microwave images. On the other hand, in the proposed scheme, standing waves within a microwave cavity are used for transmission and detection of microwave signals. A standing wave is formed when two traveling waves of nearly equal magnitudes propagate in opposite directions and each specific mode provides power distribution independent of the distance from the surface of lossy test medium. A similar phenomenon can be observed in a household microwave oven where heating is applied almost uniformly within food materials. The reason for such uniform heating in lossy food materials is that a standing wave is formed within the cavity. Of course, the microwave power level in microwave imaging will be so small that microwave exposure is rendered harmless to patients. Indeed, the radiation exposure is expected to be less than that of a typical cell phone. Hence, this scheme can resolve the attenuation issue of the scattered traveling waves on other imaging techniques while a fine resolution can be achieved regardless of the operation frequency. In fact, the entire sample area can be smaller than a wavelength.

The intended scheme is to measure fields at the resonant frequency on the surface of the microwave cavity surrounding the breast, be it phantom or real. It is well-known on the basis of the equivalence theorem that, given the surface field distribution, the accompanying field within is uniquely determined once the electromagnetic (EM) properties of the material within the enclosed volume are available. Since the material properties are yet to be evaluated, we need multiple sets of data points to determine the distribution of dielectric constant and conductivity in a lossy test sample. Such data collection is achieved by setting all possible combinations of on and off states of the antennas placed on the cavity surface. Multiple sets of equations based on a finite difference method (FDM) or other numerical techniques are established for the material characteristics with the collected surface field distributions, which are to be rapidly solved with a portable computing system. In the proposed imaging method, the resolution does not depend on the wavelength but rather the amount of data points that are closely related to the number of antennas. In principle, the resolution can be as small as needed regardless of the operation frequency.

### Technical Innovation

The proposed scheme is a diagnostic method for breast cancer that uses non-ionizing microwave. The main advantage of microwave imaging is to give minimal side effect to cancer patients compared to the traditional imaging techniques such as x-ray computed tomography (CT) and magnetic resonance imaging (MRI). However, there are two issues yet to be resolved for more widespread use of microwave in medical applications: microwave penetration into lossy biological medium and resolution.

Conventional microwave imaging relies on traveling waves to detect signals scattered from a test sample where the resolution has to be much smaller than a wavelength. Thus, in order to have good resolution, the operation frequency has to be raised. But at high frequencies, the wave penetration into the lossy biological medium becomes small. In other words, there are two conflicting conditions to be satisfied in microwave imaging. The proposed project will demonstrate feasibility for microwave imaging by overcoming those fundamental shortcomings of the current microwave technology, such as resolution and penetration into lossy tissues. In the proposed, the resolution does not depend on the operation frequency while the fields are more or less uniform within the lossy sample. The resolution can be as small as needed.

The solution to the challenging problems is use of a standing wave in a cavity that contains the test sample, as in a microwave oven, which does not have to meet such limitations. In fact, the entire cavity can be smaller than the wavelength. Of course, in the imaging process, a very low level of microwave power is needed, being less than that of a typical cell phone. Moreover, the time required for data collection will be brief, in a matter of seconds, which will provide comfort to patients compared to other currently available imaging techniques. The resulting images will show not only variation of the complex dielectric constant but also the electric conductivity of the tissue material, revealing blood vessels around tumorous areas. Such detailed images will help doctors in early cancer detection without adverse side effects to patients. Moreover, the hardware of the proposed device is relatively inexpensive and compact enough to be portable in a typical doctor's office.

The 3D images of the proposed device will be as clear as or even better than those of more expensive technologies of x-ray CT or MRI while the proposed microwave image method does not have any harmful side effect and the unit cost for purchase/service is affordable not only for breast cancer but other forms of tumor.

### Procedures

The proposed device demonstrates the capability of a novel microwave imaging technique for detecting cancerous regions well inside the body cavity with minimal microwave power, particularly for diagnosis of breast cancer, thus overcoming the most serious problem of microwave attenuation in lossy tissue that is encountered in conventional microwave imaging techniques. In addition, the resolution of the 3D can be as small as needed. Moreover, the intended proposed device will have relatively simple hardware and software of the imaging device at a low cost and be portable once fully developed.

When the field distribution on the cavity surface is available, the fields within the cavity are determined according to the equivalent theorem assuming the material properties are given. In the proposed imaging procedure, the physical parameters, such as complex dielectric constant and conductivity, of the test material within a cavity are evaluated with a number of measured field distributions on the cavity surface at different settings.

In order to measure the surface fields of the proposed cavity, microstrip antennas are placed on the inner surface of the cavity where a coaxial feed is used to collect transmission/reception at each antenna to the outside of the cavity. For each set of measurements, there will be only one or more antennas to be excited but only one antenna is open for measurement of the transmitted signal while all others are terminated with impedance-matched loads. At a given value of the transmitted power and phase at an antenna terminal, the fields at the open edges on the microstrip patch on the inner cavity surface are uniquely given according to the cavity model while all other areas are covered by conducting surfaces, thus providing the surface field distribution. A numerical technique of finite difference method (FDM) or other suitable technique is employed for the numerical analysis.

Since a resonant standing wave is excited, the field distribution within the cavity is relatively uniform and we will not see any issue related to penetration of microwave into a lossy tissue normally encountered in conventional microwave imaging techniques with traveling waves. On the other hand, the resolution does not depend on the wavelength at the operation frequency, but rather the number of field values on the cavity boundary surface. Generally, there are two ways to increase the number of data points: One simple way is to increase the number of antennas. And the other way is to use multiple radio frequency (RF) sources. A large number of antennas will increase the construction unit cost. It may be preferable to have synchronized multiple feeds with a reasonable number of antennas to increase the resolution.

With the multiple feeds, the number of the collected data points is the same as all possible combinations of the feed and output ports, L=N(N−1) . . . (N−M) where N and M are the numbers of antennas and feeds, respectively. For example, when N=20 and M=4, L=1,860,480, which will give a resolution of 0.8 mm for a typical volume of 10 cm×10 cm×10 cm, which would be sufficient for breast cancer detection at its early stage. If better resolution is needed, the number of antennas/feeds in hardware can be increased, or more dense grids can be put around a suspect cancerous region in the numerical analysis.

For such high number of data to be collected, the manual data collection is not feasible in practice. High-speed electronic switches will be used to collect the data within a short time.

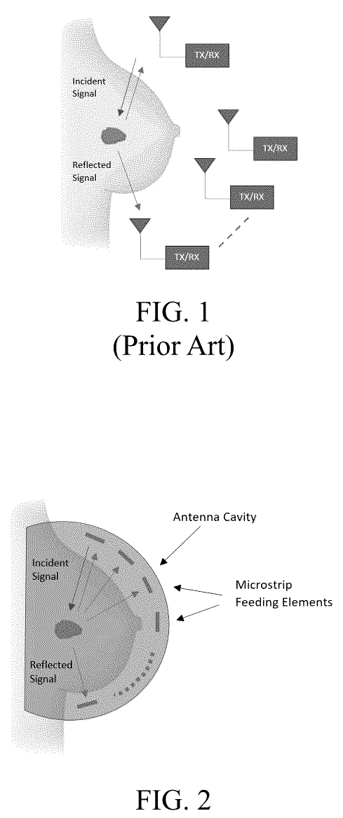

The proposed imaging method is a technique where the reported limitations can be bypassed by using standing waves in an enclosed volume as shown in FIGS. 1 and 2 in comparison with a typical radar-based microwave imaging technique that is relying on traveling waves.

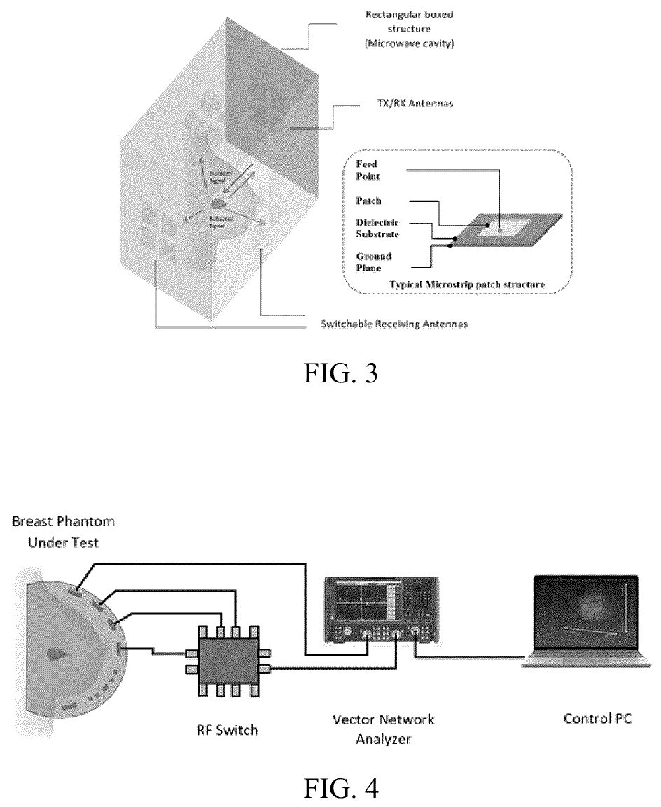

Device 1: The first device validates the proposed concept in a simple manner. To achieve such a goal, microwave imaging in a boxed structure may be considered, as shown in FIG. 3.

The test sample consists of materials with known properties that are closely related to the typical breast tissue and easily identifiable. Meat products are specifically arranged for the experiment. As an example setup, there are five sets of transmitting/receiving antennas on the five rectangular surfaces where each set contains four microstrip antennas, thus providing a total of 20 antennas. At this simple set, switching between the transmitting and receiving antennas can be performed manually. The following procedures will be followed:
Step 1 (microstrip antenna design): Design a microstrip that operates at a frequency where the fields are confined within the box cavity. At the resonant frequency, the fields beyond the open side toward the patient will be evanescent. A microstrip antenna consists of four prominent features: ground plane, insulating substrate, radiating patch on the substrate, and coaxial feed as shown in FIG. 3. The ground plane shields the cavity from the outer area. The radiating patches are placed on the inner surface of the shielding boundary. Finally, the coaxial feed lines are outside the ground surface for transmission and reception.
Step 2 (establishment of resonant structure): Connect the input RF power to one of the 20 antennas, while all others are terminated with an impedance-matched load of 50 ohms. Slide the conducting front plate facing the patient until the cavity resonates with a reasonable Q value, which indicates the interaction between the RF wave and the sample materials.
Step 3 (data collection): Once the resonant structure is selected, the next step is data collection for image processing as follows: only one antenna is used as a transmitter and another as a receiver while all other antennas are terminated with impedance matched loads such as 50-ohm terminators. For each transmitter, there will be 19 outputs, each of which contains the magnitude and phase, giving 380 data points.
Step 4 (data processing): When the material properties are available, the field distributions within the cavity are uniquely determined according to the uniqueness theorem. Microstrip antennas are placed on the inner surface of the cavity where a coaxial feed is used for transmission/reception at each antenna. For each set of measurements, there will be only one antenna to be excited and only one antenna is open for measurement of the transmitted power and phase while all others are terminated with impedance-matched loads. At a given value of the transmitted signal at an antenna terminal, the fields at the open edges on the microstrip patch on the inner cavity surface are uniquely given according to the cavity model while all other areas are covered by conducting surfaces, thus providing the surface field distribution. With N antennas, there are N data sets, each of which contains N data points (N−1 transmission coefficients and 1 reflection coefficient) for the magnitudes and phases of the field distribution at the open edges of the corresponding microstrip antenna. The cavity space is discretized into a number of points for the FDM algorithm or other suitable algorithm where each subspace within the sample region is specified by a complex dielectric constant and conductivity yet to be evaluated. The current problem in the imaging procedure is to determine the physical parameters with N sets of data, which will be numerically determined. For the current setup of 20 antennas, the cavity space is divided into a number of subspaces, 400 subspaces of which are within the test sample with unknown values of permittivity and conductivity. A matrix of 400×400 will be solved to determine the material parameters, based on a numerical technique of the finite difference method (FDM) or other suitable numerical method.
Step 5 (image processing and validation): From the processed data, a 3D image will be created and compared with the sample setup to validate the proposed concept.

Device 2: With a more refined setup as shown in FIG. 4, an improved resolution results with an electronic switching system for fast data collection. As the number of sampling points increases, the manual data collection as in Device 1 above is not feasible in practice. High-speed electronic switches will be used to collect the data within a short time. Please note that the resolution does not depend on the wavelength at the operation frequency, but rather the number of field values on the cavity boundary surface. Generally, there are two ways to increase the number of data points: one simple way is to increase the number of antennas. And the other way is to use multiple RF sources. In Device 2, both the synchronized multiple feeds and added antennas are considered for improved resolution. The specific steps to follow are listed below:
Step 1 (addition of more microstrip antennas and electronic tuning): Similar procedures as in Device 1 above will be followed to produce 3D images from the collected data except that more microstrip antennas are added to improve the resolution. Also, for the resonant frequency, electronic tuning can be installed for convenience.
Step 2 (installation of electronic switches): Since the subject sample in practice is a living being, the data has to be collected as fast as possible. Such rapid data collection is possible only with high-speed electronic switches as shown in FIG. 4. At a particular time of measurement, all the switches are turned off except for one or more transmitters and one receiver. The magnitudes and phases at all possible combinations of switch states are measured in rapid succession. In this procedure, both hardware and software are developed for rapid data collection.
Step 3 (high resolution on a focused area): Resolution near a suspect region is improved by putting more discretized points around the area of interest. Thus, the initial scan provides an overview, and subsequent scans will focus on the possible cancerous area.
Step 4 (dual RF sources): Multiple RF sources are implemented and the performance enhancements on resolution is assessed. For a single feed with N antennas, data points of N(N−1) may be collected. The problem here is the hardware becomes more complex and expensive. The second option is to have more antenna ports connected to synchronized RF sources while the number of antennas remains the same. Depending on the number of RF sources, the amount of collected data will drastically increase. For example, if M RF sources are synchronized to one another with N antennas, there will be N(N−1) . . . (N−M) measured values. For example, when N=20 and M=2, the number of data points on the boundary surface becomes 3,420. If a phase variation between the two feeds is included, there will be a large multiplication factor to the amount of the data. For example, with N=20 and the phase differences between the two sources with an increment of 45 degrees, the multiplication factor is 8 and the total number of collected value grows to 27,360.

### Key Advantages in Summary

Resolution Improvement: In conventional microwave imaging methods based on traveling waves, the resolution depends on the wavelength of operation frequency. Since the penetration depth into the lossy biological tissue is severely limited at high frequencies, the resolution of the conventional imaging techniques is not sufficient for breast cancer detection that requires a resolution of mm range or better. In the proposed scheme, drastic resolution improvement is achieved by placing a number of antennas on the cavity surface. The resolution of the proposed method does not depend on the wavelength, rather the number of collected data points that are related to the number of antennas around the sample. With a reasonable number of antennas and space-discretization algorithm with one or two RF sources, the proposed system is capable to detect a tumor size of 1 mm or less, that may detect most of breast cancer lesions.

Computational Processing Enhancement: A well-known numerical technique of finite difference method (FDM) is used for the numerical analysis/processing. The FDM procedure is versatile enough for a machine operator to focus on suspect areas by allocating dense discretization points around the region of interest. On the other hand, other numerical techniques such as finite element method (FEM) can be used for better results.

Mobility Option: The proposed design is utilizing microstrip array antennas which are compact and low profile. Such attractive features make the proposed system so small that the device can fit in a typical doctor's office. A rapid electronic measurement system is installed such that data collection be completed in a matter of seconds. Consequently, patients will be able to receive image scanning during a typical doctor's visit.

Cost Reduction: The proposed product will produce images at a significantly lower cost than those of most current imagining systems because the hardware is relatively inexpensive and the data/image processing can be completed by a high-end personal computer.

Health & Safety: The proposed solution is using microwave signal which is classified as non-ionized signal along with a very low microwave power level. Hence, microwave exposure is rendered harmless to patients.

Other Applications: In this application, the imaging device for breast cancer detection is emphasized as an illustration. But the same principles can be applied to medical imaging systems for other diagnostic tools such as for lung cancers, brain tumors, body injuries, etc.

Having thus described the present invention by reference to certain of its preferred embodiments, it is noted that the embodiments disclosed are illustrative rather than limiting in nature and that a wide range of variations in, modifications of, changes to, and substitutions of antennas are contemplated in the foregoing disclosure and, in some instances, some features of the present invention may be employed without a corresponding use of the other features. Many such variations and modifications may be considered obvious and desirable by those skilled in the art based upon a review of the foregoing description of preferred embodiments. Accordingly, it is appropriate that the appended claims be construed broadly and in a manner consistent with the scope of the invention.

1. A system for microwave imaging, the system comprising:
a conducting front plate;
a cavity defined in the conducting front plate;
microwave imaging in a boxed structure (cavity);
an array of transmitting and receiving antennas;
a microstrip antenna;
the microstrip antenna comprising a ground plane, an insulating substrate, a radiating patch on the substrate, and a coaxial feed line; and
wherein the coaxial feed line is outside the ground plane for transmission and reception.

2. The system of claim 1 wherein the microstrip antenna comprises an array of antennas configured for collecting data for image processing; wherein one antenna is used as a transmitter and another antenna as a receiver, while all other antennas are terminated.
